(19) United States
(12) Patent Application Publication
(10) Pub. No.: US 2026/0024641 A1
(43) Pub. Date: Jan. 22, 2026

(54) System and Method to Determine Parameters for a Medical Injection

(71) Applicants: Thomas J. Shaw, Frisco, TX (US); Retractable Technologies, Inc., Little Elm, TX (US)

(72) Inventor: Thomas J. Shaw, Frisco, TX (US)

(21) Appl. No.: 19/271,209

(22) Filed: Jul. 16, 2025

Related U.S. Application Data
(60) Provisional application No. 63/672,518, filed on Jul. 17, 2024.

(51) Int. Cl. G16H 20/17; G16H 30/40; G16H 40/63

(52) U.S. Cl. CPC G16H 20/17; G16H 30/40; G16H 40/63

(57) ABSTRACT

Systems and methods are disclosed that include determining medical treatment parameters based on a desired appearance. The system includes a camera, a display, and a processing system. The system may also include a projection device or system. The camera may capture one or more digital images of a body part of a subject. The display may display one of the digital images captured by the camera and allow inputs through a touchscreen user interface that alter a characteristic of the body part of the subject based on a desired appearance of the subject. The processing system may analyze the inputs and the desired appearance of the subject to calculate a medical treatment designed to achieve or to correspond to the desired appearance in the subject.

### CROSS-REFERENCE TO RELATED APPLICATIONS

This application claims priority under 35 U.S.C. § 119 (c) to U.S. Provisional Patent Application No. 63/672,518 filed on Jul. 17, 2024 by Thomas J. Shaw and entitled “System and Method To Determine Parameters For a Medical Injection,” the disclosure of which is hereby incorporated by reference in its entirety for all purposes.

### BACKGROUND

Medical injections are administered for many different purposes. Recently, cosmetic injections, such as botulinum neurotoxin injections and other injections that correct skin defects, wrinkles, or the like and/or that cosmetically enhance particular features have become increasingly popular. These injections are often administered intradermally into the lower layers of the skin and require a precision penetration depth to ensure the injection is given at an adequate depth in order to reach the desired tissue for the injection and also to ensure that the injection is not administered at too deep of a depth, which could result in additional pain to a patient, delayed or slow absorption of the medicinal fluid. These injections must also be administered at the proper dosage, location, depth, and angle in an effort to achieve the desired effect. However, the correct dosage, location, depth, and angle is often difficult to determine, sometimes requires experimentation, and must be administered by experienced, trained, and/or licensed personnel. For these reasons, the cosmetic medical industry continues to demand advances in technology that aid medical professionals in determining the proper dosage, location, depth, and angle of the injection to achieve a desired result for the patient.

### SUMMARY

Embodiments of a system for determining parameters of a medical treatment based on a desired appearance may include: a camera system configured to capture a digital image of a body part of a subject; a display configured to display the digital image captured by the camera; and a processing system configured to receive one or more inputs that alter a characteristic of the body part of the subject based on a desired appearance of the subject and calculate a medical treatment based on the one or more inputs that are designed to achieve or correspond to the desired appearance in the subject.

Embodiments of a system for determining parameters of a medical treatment based on a desired appearance may include: providing a system including a camera, a display, and a processing system; capturing a digital image of a particular body part of a subject with the camera; displaying the digital image of the subject on the display; providing one or more inputs that alter one or more characteristics of the body part of the subject based on a desired appearance of the subject; calculating a medical treatment that is designed to achieve or correspond to the desired appearance in the subject with the processing system; and displaying parameters of the medical treatment on the display.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows a simplified schematic of a system for determining parameters of a medical injection configured in a capture mode according to an embodiment of the disclosure.

FIG. 2 shows another simplified schematic of the system for determining parameters of a medical injection configured in a projection mode according to an embodiment of the disclosure.

FIG. 3 shows a simplified schematic of a processing system according to an embodiment of the disclosure.

FIG. 4 shows a flowchart of a method of determining medical treatment parameters based on a desired appearance according to an embodiment of the disclosure.

The use of the same reference symbols in different drawings indicates similar or identical items.

### DETAILED DESCRIPTION

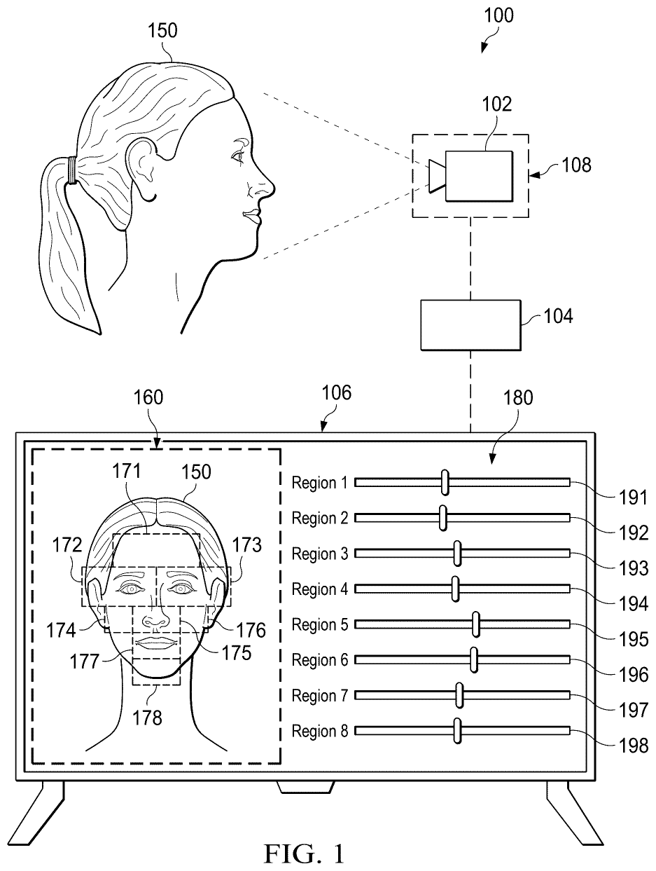

Referring to FIG. 1, a simplified schematic of a system 100 for determining parameters of a medical injection is shown according to an embodiment of the disclosure. The system 100 may generally comprise a camera 102, a processing system 104, and a display 106. In some embodiments, the system 100 may also comprise a projection device 108. In operation, the camera 102, the processing system 104, the display 106, and/or the projection device 108 may be coupled in wired and/or wireless communication for the transfer of digital images and data such as user inputs, medical data, and treatment data, characteristics, or parameters, or the like.

The camera 102 may generally comprise a digital imaging device capable of capturing digital images. More specifically, the camera 102 may be capable of capturing digital images of a particular body part of a subject 150. The camera 102 may also comprise the capability of storing and transferring the digital images of the subject 150 to the processing system 104 and/or the display 106. In some embodiments, the camera 102 may be a dedicated or standalone component. In some embodiments, the camera 102 may be embedded in a smart phone, a tablet, the processing system 104, or the display 106.

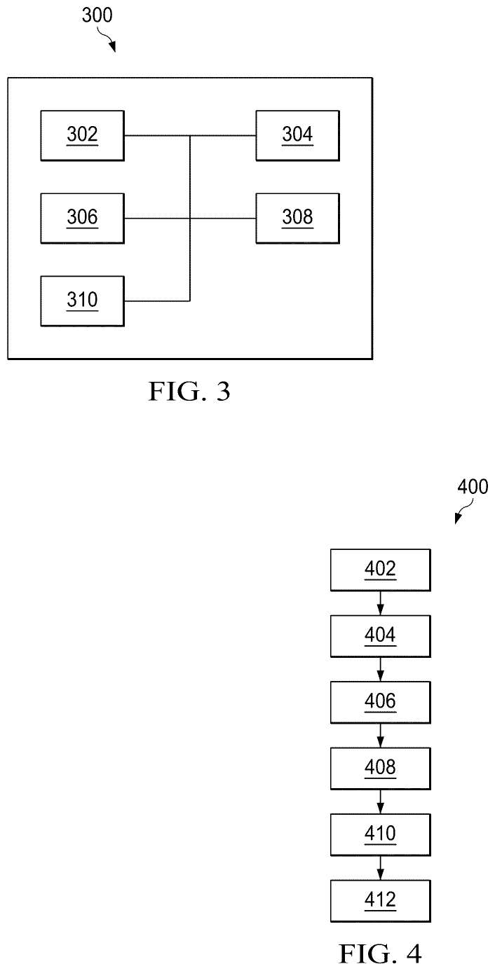

The processing system 104 may generally comprise one or more processing or control components suitable for executing instructions for implementing the embodiments disclosed herein. The processing system 104 may also comprise additional components, such as memory, storage, one or more network connectivity devices or communications interfaces, and/or one or more user interfaces that cooperate with the one or more processing or control components, which are described in more detail with respect to FIG. 3. In some embodiments, the processing system 104 may control the camera 102 and/or the display 106. In some embodiments, the processing system 104 may be coupled to or embedded in the camera 102. In some embodiments, the processing system 104 may be coupled to or embedded in the display 106. In some embodiments, the processing system 104 may have components embedded in each of the camera 102 and the display 106. In other embodiments, the camera 102, the processing system 104, and the display 106 may comprise a fully integrated, unitary system.

The display 106 may generally comprise a flat panel display. The display 106 may be capable of displaying a captured digital image of a subject 150 received from the camera 102. In some embodiments, the captured digital image may be communicated to the processing system 104 and/or the display 106. In some embodiments, the display 106 may comprise embedded software and display digital images or screens associated with or generated by the embedded software. In some embodiments, the display 106 may display digital images or screens associated with or generated by software executed by the processing system 104. In some embodiments, the display 106 may comprise a touchscreen display and comprise a user interface that accepts inputs to the processing system 104.

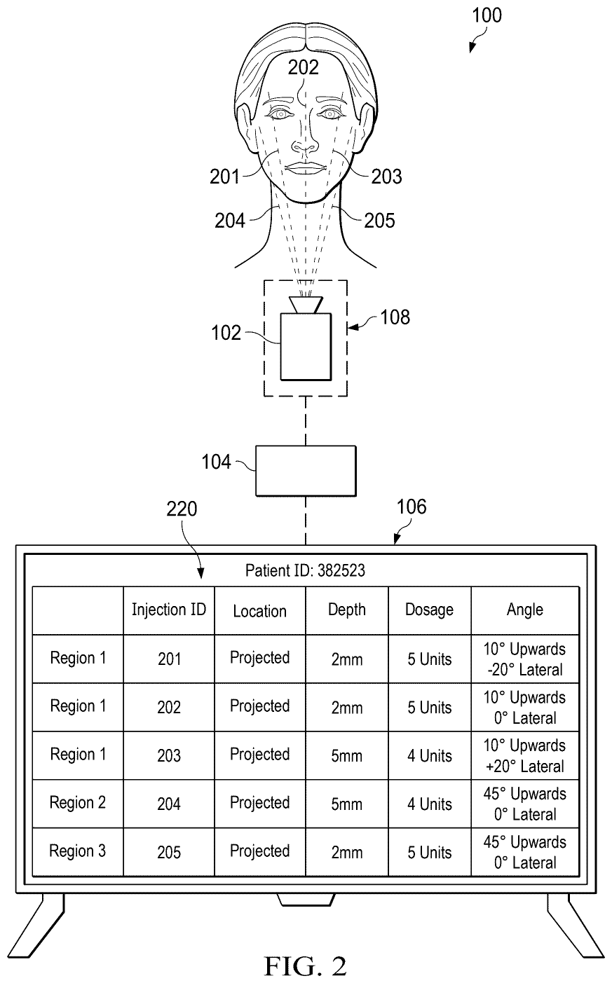

Referring to FIGS. 1 and 2, simplified schematics of the system 100 for determining parameters of a medical injection are shown. In operation, the camera 102 may generally capture digital images of a particular body part of a subject 150. For example, in the embodiment shown, the camera 102 is configured to capture a digital image of the face of a subject 150. However, in other embodiments, the camera 102 may capture a digital image of any desired body part of the subject 150 requiring or desiring medical treatment (e.g., leg, chest, etc.). In some embodiments, multiple digital images may be captured of the desired body part of the subject 150. Digital image(s) captured by the camera 102 may be conveyed to the processing system 104 and/or the display 106. One or more of the digital images may be selected for display and use on the display 106. In some embodiments, the selection may be performed by a user or subject 150 through the camera 102, the processing system 104, and/or the display 106. However, in some embodiments, a live digital image may be displayed.

The displayed digital image 160 may be displayed on the display 106 for viewing by the subject 150. The digital image 160 may be divided into a plurality of demarcated regions 171, 172, 173, 174, 175, 176, 177, 178. The demarcated regions 171, 172, 173, 174, 175, 176, 177, 178 may be transposed onto or over the displayed digital image 160. Each region 171, 172, 173, 174, 175, 176, 177, 178 may be associated with a particular feature or area of the subject 150 on the displayed digital image 160. For example, region 171 indicates the forehead of the face of the subject 150; region 172 indicates the right eye area of the face of the subject 150; region 173 indicates the left eye area of the face of the subject 150; region 174 indicates the right check area of the face of the subject 150; region 175 indicates the nose area of the face of the subject 150; region 176 indicates the left cheek area of the face of the subject 150; region 177 indicates the left mouth area of the face of the subject 150; and region 178 indicates the chin area of the face of the subject 150.

A control area 180 may also be displayed on display 106. The control area 180 may comprise an array of a plurality of individual controls 191, 192, 193, 194, 195, 196, 197, 198 that may accept adjustments or inputs from a user or subject 150. In the embodiment shown, the controls 191, 192, 193, 194, 195, 196, 197, 198 comprise sliders. However, the controls 191, 192, 193, 194, 195, 196, 197, 198 may comprise any other suitable digital input capable of being displayed and accepting adjustments or inputs from a user or subject 150. Each control 191, 192, 193, 194, 195, 196, 197, 198 may be associated with a demarcated region 171, 172, 173, 174, 175, 176, 177, 178. For example, the first control 191 may be associated with the first region 171, the second control 192 may be associated with the second region 172, and so on. Accordingly, each of the controls 191, 192, 193, 194, 195, 196, 197, 198 may accept adjustments or inputs that alters the characteristics (e.g., appearance) of the particular feature or area of the subject 150 associated with each region 171, 172, 173, 174, 175, 176, 177, 178.

The adjustments or inputs received through the controls 191, 192, 193, 194, 195, 196, 197, 198 may indicate an increase in size, decrease in size, reduction in wrinkles, reduction in hyperpigmentation, sunspots, or other skin irregularities, or any other appearance-related alteration that may be addressed by one or more medical injections or treatments. Upon adjustment or input received through one or more controls 191, 192, 193, 194, 195, 196, 197, 198, the appearance-related alteration may be displayed in the associated region 171, 172, 173, 174, 175, 176, 177, 178 of the displayed digital image 160 to show in real-time how the adjustment would alter the appearance of the subject 150. For example, if the subject 150 desires a reduction in wrinkles in region 171 associated with the forehead of the face of the subject 150, the subject 150 may adjust control 191 until the desired appearance is achieved in the displayed digital image 160. Thus, the subject 150 may continually adjust each of the controls 191, 192, 193, 194, 195, 196, 197, 198 until the desired appearance of the subject 150 is shown in the displayed digital image.

In some embodiments, to adjust a region 171, 172, 173, 174, 175, 176, 177, 178, the subject 150 may select the desired region 171, 172, 173, 174, 175, 176, 177, 178 via touchscreen selection on the displayed digital image 160. In some embodiments, when the desired region 171, 172, 173, 174, 175, 176, 177, 178 is selected, the selected region may be indicated on the displayed image by either highlighting, changing color, or the like. In some embodiments, the control 191, 192, 193, 194, 195, 196, 197, 198 associated with the selected region may also become highlighted, change color, or the like. In some embodiments, only one region 171, 172, 173, 174, 175, 176, 177, 178 may be selected and/or altered at a time. However, in some embodiments, one or more of the regions 171, 172, 173, 174, 175, 176, 177, 178 may be simultaneously selected and/or altered.

Once the subject 150 has completed adjustments and inputs through the controls 191, 192, 193, 194, 195, 196, 197, 198, and the subject's 150 desired appearance is displayed on the displayed digital image 160, the adjustments and inputs may be communicated to the processing system 104. The processing system 104 may analyze the adjustments and inputs to the displayed digital image 160 and/or the compare the subject's 150 desired appearance to the subject's 150 current appearance as captured by the camera 102. Thereafter, the processing system 104 may calculate a medical treatment that is designed to achieve or correspond to the desired appearance in the subject 150. In some embodiments, the medical treatment may include injection parameters associated with each region 171, 172, 173, 174, 175, 176, 177, 178. In some embodiments, the injection parameters may comprise an injection location, an injection depth, a medicine or pharmaceutical preparation or an implantable device or filler for an injection or insertion, or a dosage amount for the medicine or pharmaceutical preparation or filler, a size for an implantable device, and injection angle, and/or an injection identification (ID) number for each region 171, 172, 173, 174, 175, 176, 177, 178. In some embodiments, the medical treatment may comprise an implant or filler location, an implant or filler depth, or a size or type for an implantable device or filler for each region 171, 172, 173, 174, 175, 176, 177, 178. In some embodiments, a medicine or medical preparation may comprise botulinum toxin type A, onabotulinumtoxinA (such as Botox®), abobotulinumtoxinA (such as Dysport®), incobotulinumtoxinA (such as Xcomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject. In some embodiments, implants or fillers may comprise chin implants, lip implants, check implants, jaw implants, or any other implantable device suitable for cosmetic or medical use on a human subject. In some embodiments, the injection parameters may also comprise a frequency, time interval, and/or date of a subsequent medical treatment if multiple medical treatments are needed to achieve or correspond to the desired appearance in the subject 150. If no adjustments or inputs were made to one or more particular regions 171, 172, 173, 174, 175, 176, 177, 178, no medical treatment for those regions will be calculated or determined.

As shown in FIG. 2, once the processing system 104 calculates the medical treatment, the medical treatment parameters 220 may be stored by the processing system 104 and/or communicated to and displayed on the display 106. In some embodiments, the medical treatment parameters 220 may also be communicated to the projection device 108. In some embodiments, the projection device 108 may be positioned similarly to the camera 102 with respect to the subject 150. The projection device 108 may indicate the desired regions 171, 172, 173 for treatment in accordance with the medical treatment parameters 220 displayed on the display 106. In some embodiments, the projection device 108 may project a narrow beam of light or laser, or otherwise illuminate the injection locations associated with the desired regions 171, 172, 173 for treatment. In some embodiments, the projection device 108 may project a colored illumination associated with a color-coded desired region 171, 172, 173 for treatment and/or associated with a specific injection 201, 202, 203, 204, 205.

In the embodiment shown, the first region 171 will receive injections 201, 202, 203 at the projected location, the second region 172 will receive injection 204 at the projected location, and the third region will receive injection 205 at the projected location. No injections were calculated for regions 174, 175, 176, 177, 178 based on the subject's 150 desired appearance, which in this example, may be to reduce wrinkles in regions 171, 172, 173 (forehead, right eye area, left eye area) via botulinum neurotoxin injections. The medical treatment (injection) parameters 220 are displayed on the display 106 and indicate the Region, the Injection ID, the Depth, the Dosage, and the Angle for each injection 201, 202, 203, 204, 205. The medical treatment (injection) parameters 220 are reproduced in the chart below:

| | Injection ID | Location | Depth | Dosage | Angle |
|---|---|---|---|---|---|
| Region 1 | 201 | Projected | 2 mm | 5 Units | 10° Upwards |
| | | | | | −20° Lateral |
| Region 1 | 202 | Projected | 2 mm | 5 Units | 10° Upwards |
| | | | | | 0° Lateral |
| Region 1 | 203 | Projected | 5 mm | 4 Units | 10° Upwards |
| | | | | | +20° Lateral |
| Region 2 | 204 | Projected | 5 mm | 4 Units | 45° Upwards |
| | | | | | 0° Lateral |
| Region 3 | 205 | Projected | 2 mm | 5 Units | 45° Upwards |
| | | | | | 0° Lateral |

In some embodiments, the projection device 108 may illuminate a sequence of injections in accordance with the medical treatment. In some embodiments, the projection device 108 may illuminate the location for a first injection, such as injection 201, until a sensor disposed in the camera 102, the processing system 104, and/or the projection device 108 indicates that the injection has begun or been performed by a medical professional. The projection device 108 may continue to sequentially illuminate the injection locations for all subsequent injections 202, 203, 204 205 based on the medical treatment.

The system 100 may be configured to capture a digital image of a subject 150, allow the subject to adjust controls 191, 192, 193, 194, 195, 196, 197, 198 associated with one or more demarcated regions 171, 172, 173, 174, 175, 176, 177, 178 displayed or transposed on or over the displayed digital image 160 of the subject 150 to achieve a desired appearance or visualize how certain adjustments or inputs would change the subject's 150 appearance. Once the subject 150 has completed the adjustments or inputs, the desired appearance may be readily visible on the displayed digital image 160. The processing system 104 may calculate a medical treatment plan comprising one or more medical injections that are designed to achieve or correspond to the desired appearance in the subject 150. In some embodiments, the medical treatment parameters 220 of the medical treatment plan may be displayed on the display 106. In some embodiments, the projection device 108 may illuminate (e.g., by light or laser) the injection locations associated with the regions 171, 172, 173, 174, 175, 176, 177, 178 requiring the medical treatment that are designed to achieve or correspond to the desired appearance. This automated system 100 may reduce or altogether eliminate the guesswork in determining an injection location, an injection depth, an injection dosage amount, an injection location, an injection depth, injection angles, number of injections, and/or injection frequency or number of injection treatments to achieve a desired appearance for a subject 150.

Referring to FIG. 3, a simplified schematic of a processing system 300 is shown according to an embodiment of the disclosure. Processing system 300 may generally be substantially similar to processing system 104 and be capable of executing instructions related to the methods and/or operation described herein. In some embodiments, the processing system 300 may comprise a standalone component that communicates with the other components of the system 100. In some embodiments, the processing system 100 may be embedded in the camera 102, the display 106, or may have components embedded in each of the camera 102 and the display 106. In some embodiments, the camera 102, the processing system 104, and the display 106 may comprise a unitary system integrated into a unitary component. In systems 100 comprising a projection device 108, the projection device 108 may also be integrated into the unitary component.

The processing system 300 may generally comprise one or more processing or control components 302 suitable for implementing one or more embodiments disclosed herein. The system 300 may also comprise memory 304, storage 306, one or more network connectivity devices or communications interfaces 308, and/or one or more user interfaces 310. In some embodiments, one or more of these components may not be present or may be combined in various combinations with one another or with other components not shown. These components might be located in a single physical entity or in more than one physical entity. Any actions described herein as being executed by the processing or control component 302 may be executed solely by the processing or control component 302 or by the processing or control component 302 in conjunction with one or more other components.

The processing or control component 302 may generally execute instructions, codes, computer programs, or scripts that it might access from the memory 304, storage 306, or user interfaces 310. While only one processing or control component 302 is shown, multiple processing or control components 302 may be employed. Thus, while instructions may be discussed as being executed by a processor, the instructions may be executed simultaneously, serially, or otherwise by one or multiple processing or control components 302. In some embodiments, the processing or control component 302 may be implemented using an application-specific or special-purpose processing engine such as, for example, a microprocessor, controller, or other control logic.

The memory 304 may comprise random access memory (RAM) that may store information and instructions to be executed by the processing or control component 302. The memory 304 may also store transient data, computations, and computational results and may be used for input/output (I/O) buffering and graphical user interface (GUI) buffering. The storage 306 may comprise a non-volatile memory device such as a flash drive, EEPROM, read only memory (ROM), magnetic hard drive, solid state hard drive, or other drive (e.g., combination hard drive/SSD) which may operate to store instructions and/or data that is read during execution of instructions by the processing or control component 302.

The one or more network connectivity devices or communications interfaces 308 allow communication between various devices or components to enable receiving and transmitting data in connection with embodiments described herein. For example, images taken by the camera 102, parameters for the medical injections, locations to be projected by the projection device 108, and other data may be communicated between the camera 102, the processing system 104, the display 106, and the projection device. The one or more network connectivity devices or communications interfaces 308 may also enable the processing or control component 302 to communicate with the Internet, one or more telecommunications networks, or other network from which the processing or control component 302 may receive data or information or to which the processing or control component 302 may output data or information. Examples of a communications interface 308 may include a modem, a network interface (such as an Ethernet, network interface card, IEEE 802.XX, universal serial bus (USB) interface devices, wireless local area network (WLAN) devices, or other interface), a communications port (such as for example, a USB port, IR port, RS232 port Bluetooth® interface, or other port), or other communications interface. Communications may be carried through a communications channel, such as a wired or wireless communication medium. Some examples of a channel might include a phone line, a cellular link, an RF link, an optical link, a network interface, a local or wide area network, and other wired or wireless communications channels.

The user interface 310 may also comprise one or more input/output (I/O) devices, such as one or more touch screen displays, keyboards, keypads, mice, voice recognizing devices, sensors, and/or other well-known input/output devices. The user interface 310 may enable a user to interact with the system 100. The user interface 310 may also comprise one or more touchscreen displays, speech recognition devices, keyboards, keypads, mice, voice recognizing devices, sensors, and/or other well-known input/output devices configured to receive user inputs that enable interaction with software executed by the processing or control component 302. In some embodiments, the user interface 310 may be embedded into the display 106 and/or may include one or more visual displays, monitor interfaces, or remote access interfaces.

Referring to FIG. 4, a flowchart of a method 400 of determining medical treatment parameters based on a desired appearance is shown according to an embodiment of the disclosure. The method 400 may begin at block 402 by capturing a digital image of a particular body part of a subject 150. In some embodiments, this may be performed by the camera 102, which may be a digital imaging device capable of capturing digital images of a particular body part of a subject 150.

The method 400 may continue at block 404 by displaying the digital image of the subject 150 on a display 106. In some embodiments, the displayed digital image 160 may comprise one or more demarcated regions 171, 172, 173, 174, 175, 176, 177, 178 that overlie or are transposed over the displayed digital image 160. In some embodiments, the demarcated regions 171, 172, 173, 174, 175, 176, 177, 178 may be associated with a particular feature or area of the particular body part of the subject 150.

The method 400 may continue at block 406 by adjusting one or more controls 191, 192, 193, 194, 195, 196, 197, 198 that alters the characteristics (e.g., appearance) of the particular feature or area of the subject 150 associated with one or more corresponding regions 171, 172, 173, 174, 175, 176, 177, 178 that are designed to achieve or correspond to a desired appearance in the subject 150. In some embodiments, the characteristics of the particular feature or area of the subject 150 may be an increase in size, decrease in size, reduction in wrinkles, reduction in hyperpigmentation, sunspots, or other skin irregularities, or any other appearance-related alteration that may be addressed by one or more medical injections or treatments. In some embodiments, upon adjusting the one or more controls 191, 192, 193, 194, 195, 196, 197, 198, the appearance-related alteration may be displayed in the associated region 171, 172, 173, 174, 175, 176, 177, 178 of the displayed digital image 160 to show in real-time how the adjustment would alter the appearance of the subject 150.

The method 400 may continue at block 408 by calculating a medical treatment that is designed to achieve or correspond to the desired appearance in the subject 150. In some embodiments, this may be performed by the processing system 104 by analyzing the adjustments to the displayed digital image 160 and/or the comparing the subject's 150 desired appearance to the subject's 150 current appearance as captured by the camera 102. In some embodiments, the medical treatment may include injection parameters associated with each region 171, 172, 173, 174, 175, 176, 177, 178. In some embodiments, the injection parameters may comprise an injection location, an injection depth, an injection dosage amount, an injection location, an injection depth, and injection angle, and/or an injection identification (ID) number for each region 171, 172, 173, 174, 175, 176, 177, 178. In some embodiments, the injection parameters may also comprise a frequency, time interval, and/or date of a subsequent medical treatment if multiple medical treatments are needed to achieve the desired appearance in the subject 150. If no adjustments or inputs were made to one or more particular regions 171, 172, 173, 174, 175, 176, 177, 178, no medical treatment for those regions will be calculated or determined.

The method 400 may continue at block 410 by displaying the medical treatment parameters 220 on the display 106. In some embodiments, the medical treatment parameters 200 displayed on the display 106 may comprise the Region (injection location), the Injection ID, the Depth, the Dosage, and the Angle for each injection 201, 202, 203, 204, 205.

The method 400 may optionally continue at block 412 by illuminating (e.g., by light or laser) one or more injection locations associated with the regions 171, 172, 173, 174, 175, 176, 177, 178 requiring the medical treatment on the subject that are designed to achieve or correspond to the desired appearance. In some embodiments, this may be performed by the projection device 108. In some embodiments, the projection device 108 may illuminate a sequence of injections in accordance with the medical treatment. In some embodiments, the projection device 108 may illuminate the location for a first injection, such as injection 201, until a sensor disposed in the camera 102, the processing system 104, and/or the projection device 108 indicates that the injection has begun or been performed by a medical professional. The projection device 108 may continue to sequentially illuminate the injection locations for all subsequent injections 202, 203, 204 205 based on the medical treatment.

### Examples

It will be appreciated that Examples of a system 100 or method 400 of determining medical treatment parameters based on a desired appearance may include one or more of the following Examples:
Example 1. A system for determining medical treatment parameters based on a desired appearance for a body part of a subject, comprising: a camera system configured to capture one or more digital images of the body part of the subject; a display configured to display the one or more digital images captured by the camera system; and a processing system configured to receive one or more inputs that alter a characteristic of the body part of the subject based on the desired appearance for the body part and to calculate a medical treatment based on the one or more inputs that correspond to the desired appearance for the body part.
Example 2. The system of Example 1, wherein the camera system, the processing system, and the display are coupled in wired or wireless communication for transferring the one or more digital images and data associated with the medical treatment therebetween.
Example 3. The system of any of the preceding Examples, wherein the camera system is configured to store and transfer the one or more digital images to the processing system, the display, or a combination thereof.
Example 4. The system of any of the preceding Examples, wherein the processing system or the camera system is further configured to divide at least one of the one or more digital images displayed on the display into a plurality of demarcated regions on the display.
Example 5. The system of any of the preceding Examples, wherein the plurality of demarcated regions are transposed over the at least one of the one or more digital images displayed on the display.
Example 6. The system of any of the preceding Examples, wherein each of the plurality of demarcated regions is associated with a particular area of the body part of the subject.
Example 7. The system of any of the preceding Examples, wherein the camera system is configured to capture multiple digital images of the body part of the subject.
Example 8. The system of Example 7, wherein the digital image displayed on the display is selected from the multiple digital images of the body part of the subject.
Example 9. The system of any of the preceding Examples, wherein the at least one of the one or more digital images displayed on the display is selected through the camera system, the processing system, or the display.
Example 10. The system of any of the preceding Examples, wherein the display comprises a touchscreen user interface that accepts inputs to the processing system.
Example 11. The system of any of the preceding Examples, wherein the display displays a control area on the display with the at least one of the one or more digital images displayed on the display.
Example 12. The system of Example 11, wherein the control area comprises an array of controls that accept at least one of the one or more inputs received by the processing system from a user or the subject.
Example 13. The system of Example 12, wherein each of the array of controls is associated with a demarcated region that is transposed over the at least one of the one or more digital images displayed and associated with a particular area of the body part of the subject.
Example 14. The system of any of the preceding Examples 11-12, wherein each of the array of controls is configured to receive the one or more inputs that alter an appearance of the particular area of the body part of the subject associated with their respective regions.
Example 15. The system of any of the preceding Examples, wherein an appearance-related alteration caused by the one or more inputs is displayed on the display to show the appearance-related alteration in real-time on the displayed digital image.
Example 16. The system of any of the preceding Examples, wherein the processing system is configured to control the camera system, the display, or a combination thereof.
Example 17. The system of any of the preceding Examples, wherein the processing system is configured to receive the one or more inputs that alter the characteristic of the body part of the subject from a touchscreen user interface of the display.
Example 18. The system of any of the preceding Examples, wherein the processing system is configured to analyze the one or more inputs, compare the desired appearance to the displayed digital image, or a combination thereof to calculate the medical treatment to achieve the desired appearance in the subject.
Example 19. The system of any of the preceding Examples, wherein the medical treatment comprises injection parameters associated with each of a plurality of demarcated regions that are transposed over the at least one of the one or more digital images displayed on the display and associated with a particular area of the body part of the subject.
Example 20. The system of any of the preceding Examples, wherein the injection parameters comprise an injection location, an injection depth, an injection dosage amount, and an injection angle for each injection.
Example 21. The system of any of the preceding Examples, wherein the injection parameters comprise a frequency or time interval for a subsequent medical treatment when the processing system determines multiple medical treatments are needed to achieve the desired appearance in the subject.
Example 22. The system of any of the preceding Examples, wherein the injection parameters are stored by the processing system, communicated to the display, displayed on the display, or a combination thereof.
Example 23. The system of any of the preceding Examples, further comprising: a projection device.
Example 24. The system of any of the preceding Examples, wherein the camera, the processing system, the display, and the projection device are coupled in wired or wireless communication for transferring digital images and data associated with the medical treatment therebetween.
Example 25. The system of any of the preceding Examples, wherein the projection device is configured to illuminate one or more injection locations for treatment.
Example 26. The system of any of the preceding Examples, wherein the one or more injection locations are associated with the medical treatment.
Example 27. The system of any of the preceding Examples, wherein the projection device is configured to illuminate a sequence of injections in accordance with the medical treatment.
Example 28. The system of any of the preceding Examples, wherein the processing system is further configured to (1) modify at least one of the one or more digital images into an altered image comprising one or more appearance-related alterations based on the one or more inputs, (2) to display the altered image on the display in real time to allow a user or the subject to visualize how each of the one or more inputs changes the appearance of the body part, and (3) to calculate the medical treatment once the altered image corresponds to the desired appearance for the body part.
Example 29. The system of any of the preceding Examples, particularly Example 28, wherein the processing system is further configured to transpose over the one or more digital images or the altered image or both displayed on the display a demarcation grid comprising a plurality of intersecting lines to divide the body part in the one or more digital images or the altered image into distinct regions, and wherein a location of one or more of the plurality of intersecting lines may optionally be modify by a user or the subject.
Example 30. The system any of the preceding Examples, wherein the medical treatment calculated by the processing system comprises injection parameters comprising one or more of an injection location, an injection depth, an injection location, an injection angle, identification of a medicine or pharmaceutical preparation an injection, or a dosage amount for the medicine or pharmaceutical preparation.
Example 31. The system of any of the preceding Examples, wherein the medical treatment calculated by the processing system comprises identification of an implantable device or filler for an injection or insertion on the body part, an implant or filler location, an implant or filler depth, or a size or type for an implantable device or filler
Example 32. The system of any of the preceding Examples, wherein a medicine or medical preparation in the medical treatment comprises one or more of botulinum toxin type A, onabotulinumtoxinA (such as Botox®), abobotulinumtoxinA (such as Dysport®), incobotulinumtoxinA (such as Xcomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.
Example 33. The system of any of the preceding Examples, wherein the medical treatment comprises insertion of a chin implant, a lip implant, a check implant, a jaw implant, or insertion of any other implantable device suitable for cosmetic or medical use on a human subject.
Example 34. A method of determining medical treatment parameters based on a desired appearance for a body part of a subject, comprising: capturing a digital image of the body part of the subject with a camera; displaying the digital image on a display; receiving one or more inputs that alter one or more characteristics of the body part of the subject into a processing system; calculating a medical treatment with the processing system based on the one or more inputs to correspond to the desired appearance.
Example 35. The method of Example 34, further comprising displaying parameters of the medical treatment on the display, and wherein the camera, the display, and the processing system are coupled in wired or wireless communication for transferring digital images and data associated with the medical treatment therebetween.
Example 36. The method of any of the preceding method Examples, further comprising: storing and transferring the digital image from the camera to the processing system, the display, or a combination thereof.
Example 37. The method of any of the preceding method Examples, further comprising: dividing the displayed digital image into a plurality of demarcated regions on the display.
Example 38. The method of any of the preceding method Examples, further comprising: transposing the demarcated regions over the displayed digital image on the display.
Example 39. The method of any of the preceding method Examples, wherein each of the demarcated regions is associated with a particular area of the body part of the subject.
Example 40. The method of any of the preceding method Examples, capturing multiple digital images of the body part of the subject with the camera.
Example 41. The method of any of the preceding method Examples, wherein the displayed digital image is selected from the multiple digital images of the body part of the subject for displaying on the display.
Example 42. The method of any of the preceding method Examples, wherein the displayed digital image is selected through the camera, the processing system, or the display.
Example 43. The method of any of the preceding method Examples, wherein the display comprises a touchscreen user interface that accepts inputs to the processing system.
Example 44. The method of any of the preceding method Examples, further comprising: displaying a control area on the display with the displayed digital image.
Example 45. The method of any of the preceding method Examples, wherein the control area comprises an array of controls that accept one or more inputs from a user or the subject.
Example 46. The method of any of the preceding method Examples, wherein each of the controls is associated with a demarcated region that is transposed over the displayed digital image and associated with a particular area of the body part of the subject.
Example 47. The method of any of the preceding method Examples, wherein each of the controls is configured to receive one or more inputs that alter an appearance of the particular area of the body part of the subject associated with their respective regions.
Example 48. The method of any of the preceding method Examples, further comprising: displaying an appearance-related alteration caused by the one or more inputs on the display to show the appearance-related alteration in real-time on the displayed digital image.
Example 49. The method of any of the preceding method Examples, wherein the processing system is configured to control the camera, the display, or a combination thereof.
Example 50. The method of any of the preceding method Examples, further comprising: communicating the one or more inputs that alter the characteristic of the body part of the subject from a touchscreen user interface of the display to the processing system.
Example 51. The method of any of the preceding method Examples, further comprising: analyzing the one or more inputs, comparing the desired appearance to the displayed digital image, or a combination thereof by the processing system to calculate the medical treatment to achieve the desired appearance in the subject.
Example 52. The method of any of the preceding method Examples, wherein the medical treatment comprises injection parameters associated with each of a plurality of demarcated regions that are transposed over the displayed digital image and associated with a particular area of the body part of the subject.
Example 53. The method of any of the preceding method Examples, wherein the injection parameters comprise an injection location, an injection depth, an injection dosage amount, an injection location, an injection depth, and an injection angle for each injection.
Example 54. The method of any of the preceding method Examples, wherein the injection parameters comprise a frequency or time interval for a subsequent medical treatment when the processing system determines multiple medical treatments are needed to achieve the desired appearance in the subject.
Example 55. The method of any of the preceding method Examples, further comprising: storing the injection parameters by the processing system, communicating the injection parameters to the display, displaying the injection parameters on the display, or a combination thereof.
Example 56. The method of any of the preceding method Examples, wherein the system comprises a projection device.
Example 57. The method of any of the preceding method Examples, wherein the camera, the processing system, the display, and the projection device are coupled in wired or wireless communication for transferring digital images and data associated with the medical treatment associated with the medical treatment therebetween.
Example 58. The method of any of the preceding method Examples, further comprising: illuminating one or more injection locations for treatment with the projection system.
Example 59. The method of any of the preceding method Examples, wherein the one or more injection locations are associated with the medical treatment.
Example 60. The method of any of the preceding method Examples, further comprising: sequentially illuminating the one or more injection locations in accordance with the medical treatment.
Example 61. The method of any of the preceding method Examples, further comprising (1) modifying at least one of the one or more digital images into an altered image comprising one or more appearance-related alterations based on the one or more inputs using the processing system, (2) displaying the altered image on the display in real time to allow a user or the subject to visualize how each of the one or more inputs changes the appearance of the body part, and (3) calculating the medical treatment once the altered image corresponds to the desired appearance for the body part.
Example 62. The method of any of the preceding method Examples, particularly Example 61, further comprising transposing over the one or more digital images or the altered image or both displayed on the display a demarcation grid comprising a plurality of intersecting lines to divide the body part in the one or more digital images or the altered image into distinct regions, and optionally modifying a location of one or more of the plurality of intersecting lines by a user or the subject or the processing system.
Example 63. The method any of the preceding method Examples, wherein the medical treatment calculated by the processing system comprises injection parameters comprising one or more of an injection location, an injection depth, an injection location, an injection angle, identification of a medicine or pharmaceutical preparation an injection, or a dosage amount for the medicine or pharmaceutical preparation.
Example 64. The method of any of the preceding method Examples, wherein the medical treatment calculated by the processing system comprises identification of an implantable device or filler for an injection or insertion on the body part, an implant or filler location, an implant or filler depth, or a size or type for an implantable device or filler
Example 65. The method of any of the preceding method Examples, wherein a medicine or medical preparation in the medical treatment comprises one or more of botulinum toxin type A, onabotulinumtoxinA (such as Botox®), abobotulinumtoxinA (such as Dysport®), incobotulinumtoxinA (such as Xcomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.
Example 66. The method of any of the preceding method Examples, wherein the medical treatment comprises insertion of a chin implant, a lip implant, a check implant, a jaw implant, or insertion of any other implantable device suitable for cosmetic or medical use on a human subject.
Example 67. The method of any of the preceding method Examples, where the steps in the method are carried out using a system according to any one of Examples 1-33.

In the foregoing specification, the concepts have been described with reference to specific embodiments and examples. However, those of ordinary skill in the art appreciate that various modifications and changes can be made without departing from the scope of the invention as set forth in the claims below. Other examples that occur to those skilled in the art are intended to be within the scope of the claims if they have structural elements or involve method steps that do not differ from the literal language of the claims, or if they include equivalent structural elements or equivalent method steps with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments or examples, may also be provided in combination in a single embodiment. Conversely, various features that are, for brevity, described in the context of a single embodiment, may also be provided separately or in any subcombination. Any structural elements or arrangement of structural elements relative to each other, or method steps, or medicine or medical preparation, or implant or filler of an embodiment, or a preferred embodiment, herein may be used with any other structural elements, features, components, medicines, medical preparations, implants, fillers, or steps of other embodiments even if not specifically described with respect to that embodiment, unless such combination is explicitly excluded herein. Any structural element or arrangement of structural elements relative to each other, or method steps, or medicine or medical preparation, or implant or filler of an embodiment described as excluded with any particular embodiment, or preferred embodiment, herein may similarly be excluded with any other embodiment herein even if not specifically described with such embodiment. Further, references to values stated in ranges include each and every value within that range and include sub-ranges that overlap from one preferred range to another preferred range.

What is claimed is:

1. A system for determining medical treatment parameters based on a desired appearance for a body part of a subject, comprising:
a camera system configured to capture one or more digital images of the body part of the subject;
a display configured to display the one or more digital images captured by the camera system; and
a processing system configured to receive one or more inputs that alter a characteristic of the body part of the subject based on the desired appearance for the body part and to calculate a medical treatment based on the one or more inputs to achieve the desired appearance for the body part.

2. The system of claim 1, wherein the camera system, the processing system, and the display are coupled in wired or wireless communication for transferring the one or more digital images associated with the medical treatment therebetween.

3. The system of claim 2, wherein the camera system is configured to store and transfer the one or more digital images to the processing system, the display, or a combination thereof.

4. The system of claim 1, wherein the processing system is further configured to divide at least one of the one or more digital images displayed on the display into a plurality of demarcated regions on the display.

5. The system of claim 4, wherein the plurality of demarcated regions are transposed over the at least one of the one or more digital images displayed on the display.

6. The system of claim 5, wherein each of the plurality of demarcated regions is associated with a particular area of the body part of the subject.

7. The system of claim 1, wherein the display comprises a touchscreen user interface that accepts at least one of the one or more inputs that are received by the processing system.

8. The system of claim 7, wherein the processing system is configured to divide at least one of the one or more digital images displayed on the display into a plurality of demarcated regions transposed over the at least one of the one or more digital images displayed on the display;
wherein each of the plurality of demarcated regions is associated with a distinct area of the body part of the subject;
wherein the touchscreen user interface comprises an array of controls that accept at least one of the one or more inputs from a user or the subject; and
wherein each of the array of controls is associated with one of the plurality of demarcated regions.

9. The system of claim 8, wherein the processing system is configured to modify at least one of the one or more digital images into an altered image comprising one or more appearance-related alterations caused by the one or more inputs to show how the medical treatment calculated by the processing system would alter the appearance of the body part and to display the altered image on the display.

10. The system of claim 9, wherein the processing system is configured to generate in real time the altered image based on each of the one or more inputs received and to display the altered image in real time to allow the user or the subject to visualize how each of the one or more inputs changes the appearance of the body part.

11. The system of claim 1, wherein the processing system is configured to control the camera system, the display, or a combination thereof.

12. The system of claim 1, wherein the processing system is configured to receive the one or more inputs that alter the characteristic of the body part of the subject from a touchscreen user interface of the display and analyze the one or more inputs, compare the desired appearance to the displayed digital image, or a combination thereof to calculate the medical treatment to achieve the desired appearance in the subject.

13. The system of claim 12, wherein the medical treatment comprises injection parameters associated with each of a plurality of demarcated regions that are transposed over the at least one of the one or more digital images displayed on the display and associated with a particular area of the body part of the subject.

14. The system of claim 13, wherein the injection parameters comprise one or more of an injection location, an injection depth, an injection location, an injection angle, a medicine or pharmaceutical preparation an injection, or a dosage amount for the medicine or pharmaceutical preparation.

15. The system of claim 13, wherein the injection parameters comprise one or more injection locations, an injection depth for each of the one or more injection locations, an injection dosage amount for each of the one or more injection locations, and an injection angle for each of the one or more injection locations.

16. The system of claim 14, wherein the injection parameters further comprise a frequency or time interval for a subsequent medical treatment when the processing system determines more than one of the medical treatment is needed to achieve the desired appearance for the body part.

17. The system of claim 16, wherein the injection parameters are stored by the processing system, communicated to the display, displayed on the display, or a combination thereof.

18. The system of claim 1, further comprising: a projection device configured to illuminate one or more injection locations on the body part based on the medical treatment calculated by the processing system.

19. The system of claim 18, wherein the projection device is configured to illuminate a sequence of injections based on the medical treatment calculated by the processing system.

20. The system of claim 1, wherein the processing system is configured to (1) modify at least one of the one or more digital images into an altered image comprising one or more appearance-related alterations based on the one or more inputs, (2) display the altered image on the display in real time to allow a user or the subject to visualize how each of the one or more inputs changes the appearance of the body part, and (3) calculate the medical treatment once the altered image corresponds to the desired appearance for the body part.

21. The system of claim 20, wherein the medical treatment calculated by the processing system comprises injection parameters comprising one or more of an injection location, an injection depth, an injection location, an injection angle, a medicine or pharmaceutical preparation an injection, or a dosage amount for the medicine or pharmaceutical preparation.
